(19) United States
(12) Patent Application Publication
(10) Pub. No.: US 2022/0062185 A1
(43) Pub. Date: Mar. 3, 2022

(54) SOLID ORAL PHARMACEUTICAL COMPOSITIONS FOR CHRONOTROPIC ADMINISTRATION OF SARTANS

(71) Applicant: DPL PHARMA S.P.A., Rozzano (MI) (IT)

(72) Inventors: Massimo Pedrani, Melide (CH); Chiara Conti, Vigolo Marchese (PC) (IT); Salvatore Agostino Giammillari, Rozzano (MI) (IT); Giuseppe Maccari, Voghera (PV) (IT)

(73) Assignee: DPL PHARMA S.P.A., Rozzano (MI) (IT)

(21) Appl. No.: 17/413,013

(22) PCT Filed: Dec. 12, 2019
(86) PCT No.: PCT/IB2019/060692

§ 371 Date: Jun. 11, 2021

(30) Foreign Application Priority Data
Dec. 14, 2018 (IT) 102018000011123

(51) Int. Cl. A61K 9/28; A61K 9/20; A61K 31/4178

(52) U.S. Cl. CPC A61K 9/2866; A61K 31/4178; A61K 9/2054

(57) ABSTRACT

The present invention relates to solid oral pharmaceutical compositions for chronotropic administration of sartans, consisting of a monolithic matrix core comprising at least one low/medium viscosity hydroxypropyl methylcellulose, at least one medium/high viscosity hydroxypropyl methylcellulose, one or more methacrylic polymers or copolymers and/or cellulose acetate phthalate and/or hydroxypropyl methylcellulose acetate succinate or shellac and an outer coating of said core consisting of a layer comprising ethyl cellulose, a gastroresistant layer, or a layer comprising ethylcellulose coated in turn with gastroresistant polymers.

The present invention relates to solid oral pharmaceutical compositions for chronotropic administration of sartans, in particular losartan. The formulations according to the invention comprise the active ingredient in a core consisting of a monolithic matrix comprising at least one low/medium viscosity hydroxypropyl methylcellulose, at least one medium/high viscosity hydroxypropyl methylcellulose, one or more methacrylic polymers or copolymers and/or cellulose acetate phthalate and/or hydroxypropyl methylcellulose acetate succinate or shellac, and an outer coating of said core consisting of a layer comprising ethylcellulose, or of a gastroresistant layer or a layer comprising ethylcellulose coated in turn with gastroresistant polymers.

### PRIOR ART

The treatment of illnesses affected by circadian rhythms, such as “early morning disorders”, requires a pre-set concentration of medicament, available at scheduled times. Disorders with a specific circadian cycle exhibit a marked change in symptoms during the day, usually with peaks in the morning and a gradual reduction during the day.

The design of compositions able to release a medicament with timing suitable to ensure optimum treatment of disorders involving circadian variations requires full understanding of the absorption, distribution, metabolisation and elimination of the medicaments. Time-specific release is achieved by exploiting variations in pH and the different transit times of the medicaments in the gastrointestinal apparatus.

It is well known that gastric voiding times can be highly variable, depending on the type and amount of food eaten, and that the fasting pH remains on average between 1.2 and 3.0. Transit times range from a few minutes to a few hours.

In the small intestine, the pH tends to approach neutrality, and the transit time is more constant (about 3±1 hours), whereas in the colon, pH values can range from 5.5 to neutrality (pH 7.0-7.5), and transit times vary considerably from individual to individual, from a few hours to 24-48 hours.

Chronotherapy applied to cardiovascular medicine (CV) was introduced in the 1960s, but the crucial role of the circadian system in the pathogenesis of various CV diseases, such as hypertension, pulmonary embolism, stroke and arrhythmia, has mainly emerged in the last few years. For example, acute myocardial infarction (AMI) is most frequent in the early hours of the morning. The demonstration of significant circadian variations in physiopathological mechanisms and in the clinical manifestations of CV disease justifies the use of special drug-release technologies to maximise protection when the myocardium is at the highest risk level. According to a recent review, there seem to be about 20 “clock genes”, and approximately 8-10% of heart genes have a circadian expression. Studies conducted on healthy volunteers have found that the endothelial function exhibits a definite reduction in the early morning, whereas the blood pressure, heart rate, renin-angiotensin-aldosterone system and thrombotic tendency increase in the morning. It has emerged from recent studies that a body clock dysfunction is one of the risk factors for arteriosclerosis.

The blood pressure (BP) varies over 24 hours, with a peak in the morning. Since individuals can be classified on the basis of the daytime/night-time blood pressure ratio as “non-dippers” (<10%), “dippers” (10-20%), “extreme-dippers” (>20%) and “reverse-dippers”(<0%), it has been found that “non-dippers” are at three times the risk of CV disease. Compliance with hypertension treatment has proved better (80%) if the treatment is administered once rather than twice a day. According to the data in the literature, over 80% of patients with hypertension take their medication in the morning, but the efficacy and toxicity of many medicaments has been found to vary on the basis of the relationship between the dose regimen and the circadian rhythm of biochemical, physiological and behavioural processes. Several clinical trials have demonstrated that ACE inhibitors (ACEIs) have a different effect when taken in the morning rather than in the evening. Scientific evidence demonstrates that regulation of diurnal BP can be better achieved with ACEIs and angiotensin receptor blockers (ARBs) when taken in the evening rather than in the morning on waking. Ischaemic heart disease is distributed over the 24-hour period, with a higher risk during the early hours of the day, late afternoon and evening. Both the PK and the PD of nearly all the medicaments used to treat this disease have proved to be influenced by circadian rhythms.

The electrical properties of the heart are also affected by circadian rhythms. Anti-arrhythmia treatment seems to be most effective during the peak hours of the arrhythmia. Arrhythmogenesis seems to be suppressed during nocturnal sleep, and this can influence the efficacy of arrhythmia drugs according to their administration time.

The angiotensin II receptor antagonists more commonly known as sartans (losartan, valsartan, irbesartan, candesartan and telmisartan) are a class of antihypertensive medicaments that act on the renin-angiotensin system with a different mechanism from that of ACE inhibitors. The Renin-Angiotensin System (RAS) is one of the main regulatory systems of cardiovascular homeostasis, and plays an important role in regulating the blood pressure in both the long and the short term.

Losartan is one of the most widely used sartans. The current pharmaceutical forms of losartan are immediate-release coated tablets with dissolution profiles involving release of 100% of the active ingredient within 45 minutes.

Modified-release pharmaceutical forms of losartan were described, for example, in WO 2009/134956, WO 2011/144724 and CA 2947528.

The known formulations are based on monolithic, multi-particulate or multi-unit matrix or reservoir systems. The technologies used comprise gastroresistant retard systems; slow-release systems (simple matrices); solely pH-dependent release systems; solely pH-independent release systems; pulsatile-release systems (an immediate-release portion combined with a slow, gradual controlled-release portion with a simple matrix); extended-release systems (simple extended-release matrices); and reservoir systems involving the use of containment polymers, acting as semipermeable membranes.

The known formulations are mainly characterised by single-component systems wherein the release control effect is determined by a single type of excipient. This can lead to low precision of release of the active ingredient in the site and over time, and high variability of release both in vitro and in vivo.

There is therefore a need for sartans in general and losartan in particular to be carried over time to the specific site of action with gradual, constant and/or pulsatile release and subsequently, at a second stage, gradually and constantly, for a certain number of hours, to ensure homogeneous distribution with a reproducible release profile.

WO 2015/028972 describes controlled-release formulations of losartan containing a core with two different types of hydroxypropyl methylcellulose and an ethylcellulose coating. The formulations described are the “reservoir” type, not the matrix type, and release the medicament within 4-5 hours. According to WO 2015/028972, the release is pH-independent, but the reported results do not confirm said characteristic. Moreover, it has been demonstrated in a direct experimental comparison that the standard deviations of the releases at different times are greater than those obtainable with the formulations according to the invention, which act in a similar way at different pH values.

### DESCRIPTION OF THE INVENTION

It has now been found that the activity of sartans, especially losartan, can be efficiently modulated by reducing their frequency of administration and controlling their release in particular sites of the gastrointestinal tract, using complex matrices consisting of a combination of polymers with different characteristics.

In particular, it has been found that by combining at least two types of hydroxypropyl methylcellulose having different viscosities with methacrylic polymers or copolymers and/or cellulose resins or esters, formulations that eliminate the limitations of the previously known formulations can be prepared.

The solid oral controlled-release pharmaceutical compositions according to the invention comprise a core containing a sartan and an outer coating of said core, wherein:

a) the core containing the active ingredient consists of:
(i) a monolithic matrix containing the sartan, at least one hydroxypropyl methylcellulose having a viscosity ranging between 3 and 5000 mPa·s 2% in H₂O at 20° C., at least one hydroxypropyl methylcellulose having a viscosity ranging between 13500 and 280000 mPa·s 2% in H₂O at 20° C., at least one or more methacrylic polymers/copolymers and/or shellac, cellulose acetate phthalate, and/or hydroxypropyl methylcellulose acetate succinate; or
(ii) a monolithic matrix as defined above adjacent to an immediate-release layer comprising the sartan;

b) the coating consists of a layer comprising ethylcellulose, or of a gastroresistant layer or a layer comprising ethylcellulose which, in turn, is coated with gastroresistant polymers.

The sartan is preferably losartan.

The core can consist of a monolithic matrix (i) or a bi-layer system consisting of a monolithic matrix (i) adjacent to an immediate-release layer comprising a portion of the dose of losartan. There is consequently an immediate-release stage after gastric voiding, which triggers the effect of the medicament 4-5 hours after administration, followed by a slow release that ensures pharmacodynamic coverage throughout the day, thus optimising the therapeutic effect.

The coating consists of a layer comprising ethylcellulose or, in another embodiment of the invention, coating b) consists of a layer comprising ethylcellulose coated with gastroresistant polymers.

In yet another embodiment of the invention, the coating consists of a gastroresistant layer, preferably soluble at pH≥5.5.

The acrylic/methacrylic polymer or copolymer of matrix (i) is preferably selected from copolymers of pH-independent methacrylic esters, pH-independent ammonium alkyl methacrylate copolymers; amino alkyl methacrylate copolymers soluble up to pH 5.0, methacrylic acid copolymers soluble at pH≥5.5, methacrylic acid copolymers soluble at pH 6.0-7.0; and pH-dependent methacrylic acid copolymers soluble at pH≥7.0.

According to one embodiment of the invention, the acrylic polymers or copolymers are combined with each other or with shellac, or the latter can replace said acrylic polymers/copolymers.

The gastroresistant coating can be the conventional type, and typically comprises methacrylic acid copolymers soluble at pH≥5.5. Examples of said copolymers are available on the market (Eudragit). Preferably the use of polymethacrylate L 100/55 soluble at pH≥5.5; or the combination of polymethacrylate L100 with polymethacrylate S100 at the ratio of 1:10-10:1 (preferably 1:1); or shellac; or cellulose acetate phthalates/succinates are used. In the compositions according to the invention, the hydroxypropyl methylcellulose having a viscosity ranging between 3 and 5000 mPa·s 2% in H₂O at 20° C. constitutes 1 to 20% of the weight of the core, the hydroxypropyl methylcellulose having a viscosity ranging between 13500 and 280000 mPa·s 2% in H₂O at 20° C. constitutes 1 to 20% of the weight of the core, and the methacrylic polymer/copolymer constitutes 0.1 to 20% of the weight of the core.

Hydroxypropyl methylcellulose having a viscosity ranging between 3.0 and 5000 mPa·s 2% in H₂O at 20° C. is available on the market under the names of Methocel K3LV and K100 LV, K4M.

Hydroxypropyl methylcellulose having a viscosity ranging between 13500 and 280000 mPa·s 2% in H₂O at 20° C. is available on the market under the names of Methocel K 15M, K100 M and K200M.

Ethylcellulose is present in the core-coating layer in percentages ranging from 1% to 20% of the weight of the core; preferably 5%.

The matrix core can comprise conventional excipients such as diluents (microcrystalline cellulose, starches, sugars), binders (PVP, starches, cellulose, dextrins, maltodextrins, low-viscosity cellulose), glidants (colloidal silicas), flow agents (talc), lubricants (Mg stearate, fumaryl stearate, stearic acid) disintegrating agents (croscarmellose, sodium starch glycolate, crosslinked polyvinylpyrrolidone) and other functional excipients (waxes, polycarbophil, carbomer, glycerides).

Losartan is present in doses of 10 to 100 mg in the core, and 5 to 50 mg in the immediate-release layer.

The matrix is prepared by processes of partition and direct compression, dry granulation, compacting, wet granulation, melting and extrusion.

The resulting matrix/mini-matrix can then be coated with a gastroresistant film containing pH-dependent polymers that prevent release for at least 2 hours under acid pH conditions. The following can preferably be used for this purpose: pH-dependent methacrylic acid copolymers soluble at pH≥5.5 (L 100-55/L 30 D-55); pH-dependent methacrylic acid copolymers soluble at pH 6.0-7.0 (L 100/L 12.5); pH-dependent methacrylic acid copolymers soluble at pH≥7.0 (S 100/S 12.5/FS 30D); shellac; cellulose acetate phthalate; cellulose succinate.

At a third stage, a core coating can be applied which is alternative and/or additional to and beneath the gastroresistant coating with pH-independent polymers (ethylcellulose or hydroxypropyl methylcellulose with different viscosities), which act as membranes delaying the passage of losartan into the matrix/mini-matrix core following contact with biological fluids.

The matrix is coated with a quantity of polymer sufficient to guarantee that it remains intact in gastric and enteric juices for at least 2-4 hours before the release of the active ingredient from the core (lag time). To reduce the impact of the variability of gastric voiding times, the formulations can include a further gastroresistant coating (pH-dependent) outside the matrix core (pH-independent) and outside the cellulose film coating (pH-independent), to further delay contact between the biological fluids and the modified-release core (extended release).

In this way the system prevents early release during the stomach-jejunum transit time, initiating the modulated-release programme lasting up to 24 hours and ensuring homogenous distribution of the active ingredient in the duodenum, ileum and distal ileum and in the ascending, transverse and descending tracts of the large intestine.

The use of hydrophilic polymers with different rheological characteristics (viscosity/swelling properties) combined with pH-dependent and/or pH-independent polymers allows the release to be modulated for between 8 and 24 hours. If desired, a modified-, controlled-release core can be combined with an immediate-release layer (bi-layer and/or tri-layer matrix/mini-matrix); a system thus designed gives results of “therapeutic equivalence” or different levels of therapeutic efficacy. The release is highly uniform, as can be demonstrated by a very low RSD (Relative Standard Deviation) value at all points considered.

The invention is described in greater detail in the examples below.

### EXAMPLE 1

1 Kg of losartan is loaded into a granulator with 2 Kg of lactose monohydrate, 450 g of microcrystalline cellulose, 450 g of hydroxypropyl methylcellulose (HPMC K4M), 450 g of hydroxypropyl methylcellulose (HPMC K100M) and 9 g of polymethacrylate L100-55. The ingredients are mixed until a homogeneous dispersion of active ingredient in the matrices is obtained; 26 g of magnesium stearate and 45 g of talc are then added in sequence. The mixture is then homogenised for at least 15 minutes. This mixture is then compressed to obtain a tablet weighing 443 mg. The resulting tablets are film-coated with a gastroresistant solution/suspension based on 169.4 g of polymethacrylate L100-55, 86.1 g of talc, 29 g of titanium dioxide and 15.5 g of triethyl citrate, to obtain a tablet with a mean weight of 473 mg.

When subjected to disintegration and dissolution tests at pH 1, the tablets remain intact for at least 2 hours, with release below 1%; when subjected to the dissolution test at pH≥6.4 they exhibit the following release profile: not more than 20% after 1 hour, at pH 7.2 not more than 30% after 1 hour, and not more than 60% after 2 hours; the value must be >80% after 6 hours; and 100% after 10 hours.

### EXAMPLE 2

1 Kg of losartan is loaded into a granulator with 2 Kg of lactose monohydrate, 450 g of microcrystalline cellulose, 450 g of hydroxypropyl methylcellulose (HPMC K4M), 450 g of hydroxypropyl methylcellulose (HPMC K100M), 4.5 g of polymethacrylate L100 and 4.5 g of polymethacrylate S100. The ingredients are mixed until a homogeneous dispersion of active ingredient in the matrices is obtained; 26 g of magnesium stearate and 45 g of talc are then added in sequence. The mixture is then homogenised for at least 15 minutes. This mixture is then compressed to obtain a tablet weighing 443 mg. The resulting tablets are film-coated with a gastroresistant solution/suspension based on 84.7 g of polymethacrylate L100, 84.7 g of polymethacrylate S100, 86 g of talc, 29 g of titanium dioxide and 15.5 g of triethyl citrate, to obtain a tablet with a mean weight of 473 mg.

When subjected to disintegration and dissolution tests at pH 1, the tablets remain intact for at least 2 hours, with release below 1%; when subjected to the dissolution test at pH≥6.4 they exhibit the following release profile: not more than 1% after 1 hour, at pH 7.2 not more than 20% after 1 hour, and not more than 50% after 2 hours; the value must be >80% after 6 hours; and 100% after 10 hours.

### EXAMPLE 3

1 Kg of losartan is loaded into a granulator with 2 Kg of lactose monohydrate, 450 g of microcrystalline cellulose, 450 g of hydroxypropyl methylcellulose (HPMC K4M), 450 g of hydroxypropyl methylcellulose (HPMC K 100M) and 9 g of polymethacrylate L100-55. The ingredients are mixed until a homogeneous dispersion of active ingredient in the matrices is obtained; 26 g of magnesium stearate and 45 g of talc are then added in sequence. The mixture is then homogenised for at least 15 minutes. This mixture is then compressed to obtain a tablet weighing 443 mg. The resulting tablets are film-coated, firstly with a gastroresistant solution/suspension based on 169.4 g of polymethacrylate L100-55, 43 g of talc, 14.5 g of titanium dioxide and 7.75 g of triethyl citrate, and then with a solution/suspension of 270 g of ethylcellulose, 43 g of talc, 14.5 g of titanium dioxide and 7.75 g of triethyl citrate to obtain a tablet with a mean weight of 500 mg.

When subjected to disintegration and dissolution tests at pH 1, the tablets remain intact for at least 2 hours, with release below 1%; when subjected to the dissolution test at pH≥6.4 they exhibit the following release profile: not more than 1% after 5 hour, at pH 7.2 not more than 20% after 1 hour, and not more than 40% after 2 hours; the value must be >80% after 6 hours; and 100% after 18 hours.

### EXAMPLE 4

500 g of losartan is loaded into a granulator with 1.5 Kg of lactose and 337.5 g of microcrystalline cellulose. 225 g of hydroxypropyl methylcellulose (HPMC K4M), 225 g of hydroxypropyl methylcellulose (HPMC K100 M) and 4.5 g of polymethacrylate L 100-55 are added in sequence to the resulting mixture; the ingredients are mixed until a homogeneous dispersion of active ingredient in the matrices is obtained. 22.5 g of talc and 13 g of magnesium stearate are then added in sequence, and the mixture is then homogenised for at least 15 minutes. This mixture will form part of the first, controlled-release layer of the tablet.

500 g of losartan is loaded into a second granulator. 112.5 g of microcrystalline cellulose, 500 g of lactose monohydrate, 225 g of crospovidone, 225 g of croscarmellose, 13 g of magnesium stearate and 27 g of talc are added and homogeneously mixed. The mixture is then homogenised for at least 15 minutes. This mixture will form part of the second, immediate-release layer of the tablet. The two separate mixtures are then compressed to obtain a double-layer tablet weighing 443 mg. The resulting tablets are film-coated with a gastroresistant solution/suspension of 169.4 g of polymethacrylate L100-55, 86.1 g of talc, 29 g of titanium dioxide and 15.5 g of triethyl citrate, to obtain a tablet with a mean weight of 473 mg.

When subjected to disintegration and dissolution tests at pH 1, the tablets remain intact for at least 2 hours, with release below 1%; when subjected to the dissolution test at pH≥6.4 they exhibit the following release profile: not more than 5% after 1 hour, at pH 7.2 not more than 50% after 1 hour, and not more than 60% after 2 hours; not more than 75% after 6 hours; less than 85% after 8 hours; less than 95% after 10 hours; and 100% after 18 hours.

### EXAMPLE 5

500 g of losartan is loaded into a granulator with 1.5 Kg of lactose and 337.5 g of microcrystalline cellulose. 225 g of hydroxypropyl methylcellulose (HPMC K100 1v), 225 g of hydroxypropyl methylcellulose (HPMC K15 M), 2.25 g of polymethacrylate L100 and 2.25 g of polymethacrylate S 100 are added in sequence to the resulting mixture, and the ingredients are mixed until a homogeneous dispersion of active ingredient in the matrices is obtained. 22.5 g of talc and 13 g of magnesium stearate are then added in sequence. The mixture is then homogenised for at least 15 minutes. This mixture will form part of the first, controlled-release layer of the tablet.

500 g of losartan is loaded into a second granulator. 112.5 g of microcrystalline cellulose, 500 g of lactose monohydrate, 225 g of crospovidone, 225 g of croscarmellose, 13 g of magnesium stearate and 27 g of talc are added and homogeneously mixed. The mixture is then homogenised for at least 15 minutes. This mixture will form part of the second, immediate-release layer of the tablet. The two separate mixtures are then compressed to obtain a double-layer tablet weighing 443 mg. The resulting tablets are film-coated with a solution/suspension of 270 g of ethylcellulose, 86.1 g of talc, 29 g of titanium dioxide and 15.5 g of triethyl citrate, to obtain a tablet with a mean weight of 473 mg.

When subjected to disintegration and dissolution tests at pH 1, the tablets remain intact for at least 2 hours, with release below 1%; when subjected to the dissolution test at pH≥6.4 they exhibit the following release profile: not more than 5% after 1 hour, at pH 7.2 not more than 50% after 1 hour, and not more than 60% after 2 hours; not more than 75% after 6 hours; less than 80% after 8 hours; less than 95% after 10 hours; and 100% after 18 hours.

### EXAMPLE 6

500 g of losartan is loaded into a granulator with 1.5 Kg of lactose and 337.5 g of microcrystalline cellulose. 225 g of hydroxypropyl methylcellulose (HPMC K4M), 225 g of hydroxypropyl methylcellulose (HPMC K100 M) and 4.5 g of polymethacrylate L 100-55 are added in sequence to the resulting mixture; the ingredients are mixed until a homogeneous dispersion of active ingredient in the matrices is obtained. 22.5 g of talc and 13 g of magnesium stearate are then added in sequence. The mixture is then homogenised for at least 15 minutes. This mixture will form part of the first, controlled-release layer of the tablet.

500 g of losartan is loaded into a second granulator. 112.5 g of lactose monohydrate, 225 g of calcium phosphate, 225 g of crospovidone, 200 g of croscarmellose, 13 g of magnesium stearate and 27 g of talc are added and homogeneously mixed. The mixture is then homogenised for at least 15 minutes. This mixture will form part of the second, immediate-release layer of the tablet. The two separate mixtures are then compressed to obtain a double-layer tablet weighing 443 mg. The resulting tablets are film-coated, firstly with a solution/suspension of 270 g of ethylcellulose, 43.05 g of talc, 14.5 g of titanium dioxide and 7.75 g of triethyl citrate, and then with a gastroresistant solution/suspension of 169.4 g of polymethacrylate L 100-55, 43.05 g of talc, 14.5 g of titanium dioxide and 7.75 g of triethyl citrate, to obtain a tablet with a mean weight of 500 mg.

When subjected to disintegration and dissolution tests at pH 1, the tablets remain intact for at least 2 hours, with release below 1%; when subjected to the dissolution test at pH≥6.4 they exhibit the following release profile: not more than 3% after 1 hour, at pH 7.2 not more than 40% after 1 hour, and not more than 50% after 2 hours; not more than 70% after 6 hours; less than 80% after 8 hours; not more than 95% after 10 hours; and 100% after 18 hours.

### EXAMPLE 7

500 g of losartan is loaded into a granulator with 1 Kg of lactose monohydrate, 225 g of microcrystalline cellulose, 125 g of hydroxypropyl methylcellulose (HPMC K4M), 325 g of hydroxypropyl methylcellulose (HPMC K 100M) and 4.5 g of polymethacrylate L100-55. The ingredients are mixed until a homogeneous dispersion of active ingredient in the matrices is obtained; 13 g of magnesium stearate and 22.5 g of talc are then added in sequence. The mixture is then homogenised for at least 15 minutes. This mixture is then compressed to obtain a tablet weighing 221.5 mg. The resulting tablets are film-coated with a gastroresistant solution/suspension based on 122.9 g of polymethacrylate L100-55, 57.4 g of talc, 19.3 g of titanium dioxide and 15.4 g of triethyl citrate, to obtain a tablet with a mean weight of 243 mg.

When subjected to disintegration and dissolution tests at pH 1, the tablets remain intact for at least 2 hours, with release below 1%; when subjected to the dissolution test at pH≥6.4 they exhibit the following release profile: not more than 15% after 1 hour, at pH 7.2 not more than 30% after 1 hour, and not more than 60% after 2 hours; the value must be >80% after 6 hours; and 100% after 10 hours.

### EXAMPLE 8

500 g of losartan is loaded into a granulator with 1 Kg of lactose monohydrate, 225 g of microcrystalline cellulose, 125 g of hydroxypropyl methylcellulose (HPMC K4M), 325 g of hydroxypropyl methylcellulose (HPMC K 100M), 2.25 g of polymethacrylate L 100 and 2.25 g of polymethacrylate S 100. The ingredients are mixed until a homogeneous dispersion of active ingredient in the matrices is obtained; 13 g of magnesium stearate and 22.5 g of talc are then added in sequence. The mixture is then homogenised for at least 15 minutes. This mixture is then compressed to obtain a tablet weighing 221.5 mg. The resulting tablets are film-coated with a gastroresistant solution/suspension based on 61.45 g of polymethacrylate L100, 61.45 g of polymethacrylate S100, 57.4 g of talc, 19.3 g of titanium dioxide and 15.4 g of triethyl citrate, to obtain a tablet with a mean weight of 243 mg.

When subjected to disintegration and dissolution tests at pH 1, the tablets remain intact for at least 2 hours, with release below 1%; when subjected to the dissolution test at pH≥6.4 they exhibit the following release profile: not more than 1% after 1 hour, at pH 7.2 not more than 20% after 1 hour, not more than 40% after 2 hours; the value must be >80% after 6 hours; and 100% after 10 hours.

### EXAMPLE 9

500 g of losartan is loaded into a granulator with 1 Kg of lactose monohydrate, 225 g of microcrystalline cellulose, 125 g of hydroxypropyl methylcellulose (HPMC K4M), 325 g of hydroxypropyl methylcellulose (HPMC K 100M) and 4.5 g of polymethacrylate L100-55. The ingredients are mixed until a homogeneous dispersion of active ingredient in the matrices is obtained; 13 g of magnesium stearate and 22.5 g of talc are then added in sequence. The mixture is then homogenised for at least 15 minutes. This mixture is then compressed to obtain a tablet weighing 221.5 mg. The resulting tablets are film-coated, firstly with a solution/suspension of 27 g of ethylcellulose, 28.7 g of talc, 9.65 g of titanium dioxide and 7.7 g of triethyl citrate, and then with a gastroresistant suspension/solution based on 122.9 g of polymethacrylate L100-55, 28.7 g of talc, 9.65 g of titanium dioxide and 7.7 g of triethyl citrate, to obtain a tablet with a mean weight of 255 mg.

When subjected to disintegration and dissolution tests at pH 1, the tablets remain intact for at least 2 hours, with release below 1%; when subjected to the dissolution test at pH≥6.4 they exhibit the following release profile: not more than 1% after 1 hour, at pH 7.2 not more than 20% after 1 hour, and not more than 40% after 2 hours; the value must be >70% after 6 hours; >90% after 10 hours; and 100% after 18 hours.

### EXAMPLE 10

250 g of losartan is loaded into a granulator with 750 g of lactose monohydrate and 170 g of microcrystalline cellulose. 111 g of hydroxypropyl methylcellulose (HPMC K4M), 180 g of hydroxypropyl methylcellulose (HPMC K100 M) and 160 g of polymethacrylate L 100-55 are added in sequence to the resulting mixture. The ingredients are mixed until a homogeneous dispersion of active ingredient in the matrices is obtained, and 11 g of talc and 7 g of magnesium stearate are then added in sequence. The mixture is then homogenised for at least 15 minutes. This mixture will form part of the first, controlled-release layer of the tablet.

250 g of losartan is loaded into a second granulator. 50 g of microcrystalline cellulose, 250 g of lactose monohydrate, 110 g of crospovidone, 110 g of croscarmellose, 8 g of magnesium stearate and 22 g of talc are added and homogeneously mixed. The mixture is then homogenised for at least 15 minutes. This mixture will form part of the second, immediate-release layer of the tablet. The two separate mixtures are then compressed to obtain a double-layer tablet weighing 226 mg. The resulting tablets are film-coated with a gastroresistant solution/suspension of 122.9 g of polymethacrylate L100-55, 57.4 g of talc, 19.3 g of titanium dioxide and 15.4 g of triethyl citrate, to obtain a tablet with a mean weight of 247.5 mg.

When subjected to disintegration and dissolution tests at pH 1, the tablets remain intact for at least 2 hours, with release below 1%; when subjected to the dissolution test at pH≥6.4 they exhibit the following release profile: not more than 50% after 1 hour, at pH 7.2 not more than 60% after 1 hour, and not more than 70% after 2 hours; not more than 80% after 6 hours; not more than 85% after 8 hours; not more than 95% after 10 hours and 100% after 18 hours.

### EXAMPLE 11

250 g of losartan is loaded into a granulator with 750 g of lactose and 170 g of microcrystalline cellulose. 111 g of hydroxypropyl methylcellulose (HPMC K4M), 160 g of hydroxypropyl methylcellulose (HPMC K100 M), 10 g of polymethacrylate L 100 and 10 g of polymethacrylate S 100 are added in sequence to the resulting mixture. The ingredients are mixed until a homogeneous dispersion of active ingredient in the matrices is obtained, and 11 g of talc and 7 g of magnesium stearate are then added in sequence. The mixture is then homogenised for at least 15 minutes. This mixture will form part of the first, controlled-release layer of the tablet.

250 g of losartan is loaded into a second granulator. 50 g of microcrystalline cellulose, 250 g of lactose monohydrate, 110 g of crospovidone, 110 g of croscarmellose, 8 g of magnesium stearate and 22 g of talc are added and homogeneously mixed. The mixture is then homogenised for at least 15 minutes. This mixture will form part of the second, immediate-release layer of the tablet. The two separate mixtures are then compressed to obtain a double-layer tablet weighing 226 mg. The resulting tablets are film-coated with a gastroresistant solution/suspension of 61.45 g of polymethacrylate L100, 61.45 g of polymethacrylate S100, 57.4 g of talc, 19.3 g of titanium dioxide and 15.4 g of triethyl citrate, to obtain a tablet with a mean weight of 247.5 mg.

When subjected to disintegration and dissolution tests at pH 1, the tablets remain intact for at least 2 hours, with release below 1%; when subjected to the dissolution test at pH≥6.4 they exhibit the following release profile: not more than 1% after 1 hour, at pH 7.2 not more than 50% after 1 hour, and not more than 60% after 2 hours; not more than 70% after 6 hours; not more than 80% after 8 hours; not more than 90% after 10 hours; and 100% after 18 hours.

### EXAMPLE 12

250 g of losartan is loaded into a granulator with 750 g of lactose and 170 g of microcrystalline cellulose. 111 g of hydroxypropyl methylcellulose (HPMC K4M), 160 g of hydroxypropyl methylcellulose (HPMC K 100M) and 20 g of polymethacrylate L 100-55 are added in sequence to the resulting mixture. The ingredients are mixed until a homogeneous dispersion of active ingredient in the matrices is obtained, and 11 g of talc and 7 g of magnesium stearate are then added in sequence. The mixture is then homogenised for at least 15 minutes. This mixture will form part of the first, controlled-release layer of the tablet.

250 g of losartan is loaded into a second granulator. 50 g of microcrystalline cellulose, 250 g of lactose monohydrate, 110 g of crospovidone, 110 g of croscarmellose, 8 g of magnesium stearate and 22 g of talc are added and homogeneously mixed. The mixture is then homogenised for at least 15 minutes. This mixture will form part of the second, immediate-release layer of the tablet. The two separate mixtures are then compressed to obtain a double-layer tablet weighing 226 mg. The resulting tablets are film-coated, firstly with an aqueous solution/suspension of 12 g of gastroresistant ethylcellulose, 122.9 g of polymethacrylate L100-55, 2.87 g of talc and 7.7 g of triethyl citrate, and then with a gastroresistant solution/suspension of 122.9 g of polymethacrylate L100-55, 19.3 g of titanium dioxide, 7.7 g of triethyl citrate and 2.87 g of talc, to obtain a tablet with a mean weight of 259.5 mg.

When subjected to disintegration and dissolution tests at pH 1, the tablets remain intact for at least 2 hours, with release below 1%; when subjected to the dissolution test at pH≥6.4 they exhibit the following release profile: not more than 10% after 1 hour, at pH 7.2 not more than 50% after 1 hour, and not more than 60% after 2 hours; not more than 70% after 6 hours; not more than 80% after 8 hours; not more than 90% after 10 hours; and 100% after 18 hours.

### EXAMPLE 13

250 g of losartan is loaded into a granulator with 1.5 Kg of lactose monohydrate, 225 g of microcrystalline cellulose, 125 g of hydroxypropyl methylcellulose (HPMC K4M), 325 g of hydroxypropyl methylcellulose (HPMC K100M) and 4.5 g of polymethacrylate L100-55. The ingredients are mixed until a homogeneous dispersion of active ingredient in the matrices is obtained; 13 g of magnesium stearate and 22.5 g of talc are then added in sequence. The mixture is then homogenised for at least 15 minutes. This mixture is then compressed to obtain a tablet weighing 221.5 mg. The resulting tablets are film-coated with a gastroresistant solution/suspension based on 122.9 g of polymethacrylate L100-55, 57.4 g of talc, 19.3 g of titanium dioxide and 15.4 g of triethyl citrate, to obtain a tablet with a mean weight of 243 mg.

When subjected to disintegration and dissolution tests at pH 1, the tablets remain intact for at least 2 hours, with release below 1%; when subjected to the dissolution test at pH≥6.4 they exhibit the following release profile: not more than 15% after 1 hour, at pH 7.2 not more than 30% after 1 hour, and not more than 60% after 2 hours; the value must be >80% after 6 hours; and 100% after 10 hours.

### EXAMPLE 14

250 g of losartan is loaded into a granulator with 1.5 Kg of lactose monohydrate, 225 g of microcrystalline cellulose, 125 g of hydroxypropyl methylcellulose (HPMC K4M), 325 g of hydroxypropyl methylcellulose (HPMC K100M), 2.25 g of polymethacrylate L100 and 2.25 g of polymethacrylate 5100. The ingredients are mixed until a homogeneous dispersion of active ingredient in the matrices is obtained; 13 g of magnesium stearate and 22.5 g of talc are then added in sequence. The mixture is then homogenised for at least 15 minutes. This mixture is then compressed to obtain a tablet weighing 221.5 mg. The resulting tablets are film-coated with a gastroresistant solution/suspension based on 61.45 g of polymethacrylate L100, 61.45 g of polymethacrylate S100, 57.4 g of talc, 19.3 g of titanium dioxide and 15.4 g of triethyl citrate, to obtain a tablet with a mean weight of 243 mg.

When subjected to disintegration and dissolution tests at pH 1, the tablets remain intact for at least 2 hours, with release below 1%; when subjected to the dissolution test at pH≥6.4 they exhibit the following release profile: not more than 1% after 1 hour, at pH 7.2 not more than 30% after 1 hour, and not more than 60% after 2 hours; the value must be >80% after 6 hours; and 100% after 10 hours.

### EXAMPLE 15

250 g of losartan is loaded into a granulator with 1.5 Kg of lactose monohydrate, 225 g of microcrystalline cellulose, 125 g of hydroxypropyl methylcellulose (HPMC K4M), 325 g of hydroxypropyl methylcellulose (HPMC K 100M) and 4.5 g of polymethacrylate L100-55. The ingredients are mixed until a homogeneous dispersion of active ingredient in the matrices is obtained; 13 g of magnesium stearate and 22.5 g of talc are then added in sequence. The mixture is then homogenised for at least 15 minutes. This mixture is then compressed to obtain a tablet weighing 221.5 mg. The resulting tablets are film-coated, firstly with a solution/suspension of 120 g of ethylcellulose, 2.87 g of talc and 7.7 g of triethyl citrate, and then with a gastroresistant solution/suspension of 122.9 g of polymethacrylate L 100-55, 2.87 g of talc, 19.3 g of titanium dioxide and 7.75 g of triethyl citrate, to obtain a tablet with a mean weight of 255 mg.

When subjected to disintegration and dissolution tests at pH 1, the tablets remain intact for at least 2 hours, with release below 1%; when subjected to the dissolution test at pH≥6.4 they exhibit the following release profile: not more than 1% after 1 hour, at pH 7.2 not more than 20% after 1 hour, and not more than 40% after 2 hours; the value must be >70% after 6 hours; >90% after 10 hours; and 100% after 18 hours.

### EXAMPLE 16

125 g of losartan is loaded into a granulator with 937.5 g of lactose and 160 g of microcrystalline cellulose. 111 g of hydroxypropyl methylcellulose (HPMC K4M), 160 g of hydroxypropyl methylcellulose (HPMC K100 M) and 20 g of polymethacrylate L 100-55 are added in sequence to the resulting mixture. The ingredients are mixed until a homogeneous dispersion of active ingredient in the matrices is obtained, and 11 g of talc and 7 g of magnesium stearate are then added in sequence. The mixture is then homogenised for at least 15 minutes. This mixture will form part of the first, controlled-release layer of the tablet.

125 g of losartan is loaded into a second granulator. 60 g of microcrystalline cellulose, 312.5 g of lactose monohydrate, 110 g of crospovidone, 110 g of croscarmellose, 8 g of magnesium stearate and 22 g of talc are added and homogeneously mixed. The mixture is then homogenised for at least 15 minutes. This mixture will form part of the second, immediate-release layer of the tablet. The two separate mixtures are then compressed to obtain a double-layer tablet weighing 226 mg. The resulting tablets are film-coated with a gastroresistant solution/suspension of 122.9 g of polymethacrylate L100-55, 57.4 g of talc, 19.3 g of titanium dioxide and 15.4 g of triethyl citrate, to obtain a tablet with a mean weight of 247.5 mg.

When subjected to disintegration and dissolution tests at pH 1, the tablets remain intact for at least 2 hours, with release below 1%; when subjected to the dissolution test at pH≥6.4 they exhibit the following release profile: not more than 50% after 1 hour, at pH 7.2 not more than 60% after 1 hour, and not more than 70% after 2 hours; not more than 80% after 6 hours; not more than 85% after 8 hours; not more than 95% after 10 hours and 100% after 18 hours.

### EXAMPLE 17

125 g of losartan is loaded into a granulator with 937.5 g of lactose and 160 g of microcrystalline cellulose. 111 g of hydroxypropyl methylcellulose (HPMC K4M), 160 g of hydroxypropyl methylcellulose (HPMC K100 M), 10 g of polymethacrylate L 100 and 10 g of polymethacrylate S 100 are added in sequence to the resulting mixture. The ingredients are mixed until a homogeneous dispersion of active ingredient in the matrices is obtained, and 11 g of talc and 7 g of magnesium stearate are then added in sequence. The mixture is then homogenised for at least 15 minutes. This mixture will form part of the first, controlled-release layer of the tablet.

125 g of losartan is loaded into a second granulator. 60 g of microcrystalline cellulose, 312.5 g of lactose monohydrate, 110 g of crospovidone, 110 g of croscarmellose, 8 g of magnesium stearate and 22 g of talc are added and homogeneously mixed. The mixture is then homogenised for at least 15 minutes. This mixture will form part of the second, immediate-release layer of the tablet. The two separate mixtures are then compressed to obtain a double-layer tablet weighing 226 mg. The resulting tablets are film-coated with a gastroresistant solution/suspension of 61.45 g of polymethacrylate L100, 61.45 g of polymethacrylate 5100, 57.4 g of talc, 19.3 g of titanium dioxide and 15.4 g of triethyl citrate, to obtain a tablet with a mean weight of 247.5 mg.

When subjected to disintegration and dissolution tests at pH 1, the tablets remain intact for at least 2 hours, with release below 1%; when subjected to the dissolution test at pH≥6.4 they exhibit the following release profile: not more than 10% after 1 hour, at pH 7.2 not more than 50% after 1 hour, and not more than 70% after 2 hours; not more than 80% after 6 hours; not more than 85% after 8 hours; and 100% after 10 hours.

### EXAMPLE 18

125 g of losartan is loaded into a granulator with 937.5 g of lactose and 170 g of microcrystalline cellulose. 111 g of hydroxypropyl methylcellulose (HPMC K4M), 160 g of hydroxypropyl methylcellulose (HPMC K100 M) and 2 g of polymethacrylate L 100-55 are added in sequence to the resulting mixture. The ingredients are mixed until a homogeneous dispersion of active ingredient in the matrices is obtained, and 11 g of talc and 7 g of magnesium stearate are then added in sequence. The mixture is then homogenised for at least 15 minutes. This mixture will form part of the first, controlled-release layer of the tablet.

125 g of losartan is loaded into a second granulator. 60 g of microcrystalline cellulose, 312.5 g of lactose monohydrate, 110 g of crospovidone, 110 g of croscarmellose, 8 g of magnesium stearate and 22 g of talc are added and homogeneously mixed. The mixture is then homogenised for at least 15 minutes. This mixture will form part of the second, immediate-release layer of the tablet. The two separate mixtures are then compressed to obtain a double-layer tablet weighing 226 mg. The resulting tablets are film-coated, firstly with an aqueous solution/suspension of 120 g of ethylcellulose, 7.7 g of triethyl citrate and 2.87 g of talc, and then with a gastroresistant solution/suspension of 122.9 g of polymethacrylate L100-55, 2.87 g of talc, 19.3 g of titanium dioxide and 7.7 g of triethyl citrate, to obtain a tablet with a mean weight of 259.5 mg.

When subjected to disintegration and dissolution tests at pH 1, the tablets remain intact for at least 2 hours, with release below 1%; when subjected to the dissolution test at pH≥6.4 they exhibit the following release profile: not more than 5% after 1 hour, at pH 7.2 not more than 50% after 1 hour, and not more than 70% after 2 hours; not more than 80% after 6 hours; not more than 85% after 8 hours; not more than 90% after 10 hours; and 100% after 18 hours.

### EXAMPLE 19

1 Kg of losartan is loaded into a granulator with 450 g of lactose monohydrate, 2 Kg of microcrystalline cellulose, 450 g of hydroxypropyl methylcellulose (HPMC K4M), 450 g of hydroxypropyl methylcellulose (HPMC K 1 00M) and 9 g of polymethacrylate L100-55. The ingredients are mixed until a homogeneous dispersion of active ingredient in the matrices is obtained; 26 g of magnesium stearate and 45 g of talc are then added in sequence. The mixture is then homogenised for at least 15 minutes. This mixture is then compressed to obtain a mini-tablet weighing 44.3 mg. The resulting tablets are film-coated with a gastroresistant solution/suspension based on 169.4 g of polymethacrylate L100-55, 86.1 g of talc, 29 g of titanium dioxide and 15.5 g of triethyl citrate, to obtain a mini-tablet with a mean weight of 47.3 mg.

When subjected to disintegration and dissolution tests at pH 1, the mini-tablets remain intact for at least 2 hours, with release below 1%; when subjected to the dissolution test at pH≥6.4 they exhibit the following release profile: not more than 20% after 1 hour, at pH 7.2 not more than 30% after 1 hour, and not more than 60% after 2 hours; the value must be >80% after 6 hours; and 100% after 10 hours.

### EXAMPLE 20

1 Kg of losartan is loaded into a granulator with 450 g of lactose monohydrate, 2 Kg of microcrystalline cellulose, 450 g of hydroxypropyl methylcellulose (HPMC K4M), 450 g of hydroxypropyl methylcellulose (HPMC K100M), 4.5 g of polymethacrylate L100, and 4.5 g of polymethacrylate 5100. The ingredients are mixed until a homogeneous dispersion of active ingredient in the matrices is obtained; 26 g of magnesium stearate and 45 g of talc are then added in sequence. The mixture is then homogenised for at least 15 minutes. This mixture is then compressed to obtain a mini-tablet weighing 44.3 mg. The resulting tablets are film-coated with a gastroresistant solution/suspension based on 84.7 g of polymethacrylate L100, 84.7 g of polymethacrylate S100, 86.1 g of talc, 29 g of titanium dioxide and 15.5 g of triethyl citrate, to obtain a mini-tablet with a mean weight of 47.3 mg.

When subjected to disintegration and dissolution tests at pH 1, the mini-tablets remain intact for at least 2 hours, with release below 1%; when subjected to the dissolution test at pH≥6.4 they exhibit the following release profile: not more than 1% after 1 hour, at pH 7.2 not more than 30% after 1 hour, and not more than 65% after 2 hours; the value must be >80% after 6 hours; and 100% after 10 hours.

### EXAMPLE 21

1 Kg of losartan is loaded into a granulator with 450 g of lactose monohydrate, 2 Kg of microcrystalline cellulose, 450 g of hydroxypropyl methylcellulose (HPMC

K4M), 450 g of hydroxypropyl methylcellulose (HPMC K100M) and 9 g of polymethacrylate L100-55. The ingredients are mixed until a homogeneous dispersion of active ingredient in the matrices is obtained; 26 g of magnesium stearate and 45 g of talc are then added in sequence. The mixture is then homogenised for at least 15 minutes. This mixture is then compressed to obtain a mini-tablet weighing 44.3 mg. The tablets are film-coated, firstly with a solution/suspension of 27 g of ethylcellulose, 7.75 g of triethyl citrate and 43.05 g of talc, and then with a gastroresistant solution/suspension based on 169.4 g of polymethacrylate L100-55, 43.05 g of talc, 29 g of titanium dioxide and 7.75 g of triethyl citrate, to obtain a mini-tablet with a mean weight of 50 mg.

When subjected to disintegration and dissolution tests at pH 1, the mini-tablets remain intact for at least 2 hours, with release below 1%; when subjected to the dissolution test at pH≥6.4 they exhibit the following release profile: not more than 10% after 1 hour, at pH 7.2 not more than 50% after 1 hour, and not more than 60% after 2 hours; the value must be >80% after 6 hours; not more than 95% after 10 hours; and 100% after 18 hours.

### EXAMPLE 22

500 g of losartan is loaded into a granulator with 225 g of lactose and 1.5 Kg of microcrystalline cellulose. 225 g of hydroxypropyl methylcellulose (HPMC K4M), 225 g of hydroxypropyl methylcellulose (HPMC K100 M) and 4.5 g of polymethacrylate L 100-55 are added in sequence to the resulting mixture. The ingredients are mixed until a homogeneous dispersion of active ingredient in the matrices is obtained, and 22.5 g of talc and 13 g of magnesium stearate are then added in sequence. The mixture is then homogenised for at least 15 minutes. This mixture will form part of the first layer of the controlled-release mini-tablet.

500 g of losartan is loaded into a second granulator. 500 g of microcrystalline cellulose, 225 g of lactose monohydrate, 225 g of crospovidone, 225 g of croscarmellose, 13 g of magnesium stearate and 27 g of talc are added and homogeneously mixed. The mixture is then homogenised for at least 15 minutes. This mixture will form part of the second, immediate-release layer of the tablet. The two separate mixtures are then compressed to obtain a double-layer mini-tablet weighing 44.3 mg. The resulting tablets are film-coated with a gastroresistant solution/suspension of 169.4 g of polymethacrylate L100-55, 86.1 g of talc, 29 g of titanium dioxide and 15.5 of triethyl citrate, to obtain a mini-tablet with a mean weight of 47.3 mg.

When subjected to disintegration and dissolution tests at pH 1, the tablets remain intact for at least 2 hours, with release below 1%; when subjected to the dissolution test at pH≥6.4 they exhibit the following release profile: not more than 50% after 1 hour, at pH 7.2 not more than 60% after 1 hour, and not more than 70% after 2 hours; not more than 80% after 6 hours; not more than 85% after 8 hours; and 100% after 10 hours.

### EXAMPLE 23

500 g of losartan is loaded into a granulator with 225 g of lactose and 1 Kg of microcrystalline cellulose. 225 g of hydroxypropyl methylcellulose (HPMC K4M), 225 g of hydroxypropyl methylcellulose (HPMC K100 M), 2.25 g of polymethacrylate RL 100, 2.25 g of polymethacrylate SL 100 and 2.25 g of shellac are added in sequence to the resulting mixture. The ingredients are mixed until a homogeneous dispersion of active ingredient in the matrices is obtained, and 22.5 g of talc and 13 g of magnesium stearate are then added in sequence. The mixture is then homogenised for at least 15 minutes. This mixture will form part of the first layer of the controlled-release mini-tablet.

500 g of losartan is loaded into a second granulator. 500 g of microcrystalline cellulose, 225 g of lactose monohydrate, 225 g of crospovidone, 225 g of croscarmellose, 13 g of magnesium stearate and 27 g of talc are added and homogeneously mixed. The mixture is then homogenised for at least 15 minutes. This mixture will form part of the second, immediate-release layer of the tablet. The two separate mixtures are then compressed to obtain a double-layer mini-tablet weighing 45.15 mg. The resulting tablets are film-coated with a gastroresistant solution/suspension of 169.4 g of shellac, 83.85 g of talc, 29 g of titanium dioxide and 15.5 g of triethyl citrate to obtain a mini-tablet with a mean weight of 47.3 mg.

When subjected to disintegration and dissolution tests at pH 1, the tablets remain intact for at least 2 hours, with release below 1%; when subjected to the dissolution test at pH≥6.4 they exhibit the following release profile: not more than 50% after 1 hour, at pH 7.2 not more than 60% after 1 hour, and not more than 70% after 2 hours; not more than 80% after 6 hours; not more than 85% after 8 hours; and 100% after 10 hours.

### EXAMPLE 24

500 g of losartan is loaded into a granulator with 225 g of lactose and 1.5 Kg of microcrystalline cellulose. 225 g of hydroxypropyl methylcellulose (HPMC K100 1v), 225 g of hydroxypropyl methylcellulose (HPMC K15 M) and 45 g of polymethacrylate L 100-55 are added in sequence to the resulting mixture. The ingredients are mixed until a homogeneous dispersion of active ingredient in the matrices is obtained, and 22.5 g of talc and 13 g of magnesium stearate are then added in sequence. The mixture is then homogenised for at least 15 minutes. This mixture will form part of the first layer of the controlled-release mini-tablet.

500 g of losartan is loaded into a second granulator. 500 g of microcrystalline cellulose, 225 g of lactose monohydrate, 225 g of crospovidone, 225 g of croscarmellose, 13 g of magnesium stearate and 27 g of talc are added and homogeneously mixed. The mixture is then homogenised for at least 15 minutes. This mixture will form part of the second, immediate-release layer of the tablet. The two separate mixtures are then compressed to obtain a double-layer mini-tablet weighing 44.3 mg. The resulting tablets are film-coated with an aqueous solution/suspension of 270 g of ethylcellulose, 7.75 g of triethyl citrate and 43.05 of talc, and then with a gastroresistant solution/suspension of 169.4 g of Polymethacrylate L100-55, 43.05 g of talc, 29 g of titanium dioxide and 7.75 g of triethyl citrate until a mini-tablet with a mean weight of 50 mg is obtained.

When subjected to disintegration and dissolution tests at pH 1, the tablets remain intact for at least 2 hours, with release below 1%; when subjected to the dissolution test at pH≥6.4 they exhibit the following release profile: not more than 10% after 1 hour, at pH 7.2 not more than 50% after 1 hour, and not more than 70% after 2 hours; not more than 80% after 6 hours; not more than 85% after 8 hours; not more than 95% after 10 hours; and 100% after 18 hours.

### EXAMPLE 25

500 g of losartan is loaded into a granulator with 950 g of lactose monohydrate, 2.4 Kg of microcrystalline cellulose, 250 g of hydroxypropyl methylcellulose (HPMC K4M), 250 g of hydroxypropyl methylcellulose (HPMC K100M) and 9 g of polymethacrylate L100-55. The ingredients are mixed until a homogeneous dispersion of active ingredient in the matrices is obtained; 26 g of magnesium stearate and 45 g of talc are then added in sequence. The mixture is then homogenised for at least 15 minutes. This mixture is then compressed to obtain a mini-tablet weighing 44.3 mg. The resulting tablets are film-coated with a gastroresistant solution/suspension based on 169.4 g of polymethacrylate L100-55, 86.1 g of talc, 29 g of titanium dioxide and 15.5 g of triethyl citrate, to obtain a mini-tablet with a mean weight of 47.3 mg.

When subjected to disintegration and dissolution tests at pH 1, the mini-tablets remain intact for at least 2 hours, with release below 1%; when subjected to the dissolution test at pH≥6.4 they exhibit the following release profile: not more than 20% after 1 hour, at pH 7.2 not more than 30% after 1 hour, and not more than 60% after 2 hours; the value must be >80% after 6 hours; and 100% after 10 hours.

### EXAMPLE 26

500 g of losartan is loaded into a granulator with 950 g of lactose monohydrate, 2.4 Kg of microcrystalline cellulose, 250 g of hydroxypropyl methylcellulose (HPMC K4M), 250 g of hydroxypropyl methylcellulose (HPMC K100M), 4.5 g of polymethacrylate L100 and 4.5 g of polymethacrylate 5100. The ingredients are mixed until a homogeneous dispersion of active ingredient in the matrices is obtained; 26 g of magnesium stearate and 45 g of talc are then added in sequence. The mixture is then homogenised for at least 15 minutes. This mixture is then compressed to obtain a mini-tablet weighing 44.3 mg. The resulting tablets are film-coated with a gastroresistant solution/suspension based on 84.7 g of polymethacrylate L100, 84.7 g of polymethacrylate S100, 86.1 g of talc, 29 g of titanium dioxide and 15.5 g of triethyl citrate, to obtain a mini-tablet with a mean weight of 47.3 mg.

When subjected to disintegration and dissolution tests at pH 1, the mini-tablets remain intact for at least 2 hours, with release below 1%; when subjected to the dissolution test at pH≥6.4 they exhibit the following release profile: not more than 1% after 1 hour, at pH 7.2 not more than 30% after 1 hour, and not more than 65% after 2 hours; the value must be >80% after 6 hours; and 100% after 10 hours.

### EXAMPLE 27

500 Kg of losartan is loaded into a granulator with 950 g of lactose monohydrate, 2.4 Kg of microcrystalline cellulose, 250 g of hydroxypropyl methylcellulose (HPMC K4M), 250 g of hydroxypropyl methylcellulose (HPMC K 100M) and 9 g of polymethacrylate L100-55. The ingredients are mixed until a homogeneous dispersion of active ingredient in the matrices is obtained; 26 g of magnesium stearate and 45 g of talc are then added in sequence. The mixture is then homogenised for at least 15 minutes. This mixture is then compressed to obtain a mini-tablet weighing 44.3 mg. The resulting tablets are film-coated, firstly with a solution/suspension of 27 g of ethylcellulose, 7.75 g of triethyl citrate and 43.05 g of talc, and then with a gastroresistant solution/suspension based on 169.4 g of polymethacrylate L100-55, 43.05 g of talc, 29 g of titanium dioxide and 7.75 g of triethyl citrate, to obtain a mini-tablet with a mean weight of 50 mg.

When subjected to disintegration and dissolution tests at pH 1, the mini-tablets remain intact for at least 2 hours, with release below 1%; when subjected to the dissolution test at pH≥6.4 they exhibit the following release profile: not more than 10% after 1 hour, at pH 7.2 not more than 50% after 1 hour, and not more than 60% after 2 hours; the value must be >80% after 6 hours; not more than 95% after 10 hours; and 100% after 18 hours.

### EXAMPLE 28

250 g of losartan is loaded into a granulator with 475 g of lactose and 1.8 Kg of microcrystalline cellulose. 125 g of hydroxypropyl methylcellulose (HPMC K4M), 125 g of hydroxypropyl methylcellulose (HPMC K100 M) and 45 g of polymethacrylate L 100-55 are added in sequence to the resulting mixture. The ingredients are mixed until a homogeneous dispersion of active ingredient in the matrices is obtained, and 22.5 g of talc and 13 g of magnesium stearate are then added in sequence. The mixture is then homogenised for at least 15 minutes. This mixture will form part of the first layer of the controlled-release mini-tablet.

250 g of losartan is loaded into a second granulator. 600 g of microcrystalline cellulose, 475 g of lactose monohydrate, 225 g of crospovidone, 225 g of croscarmellose, 13 g of magnesium stearate and 27 g of talc are added and homogeneously mixed. The mixture is then homogenised for at least 15 minutes. This mixture will form part of the second, immediate-release layer of the tablet. The two separate mixtures are then compressed to obtain a double-layer mini-tablet weighing 44.3 mg. The resulting tablets are film-coated with a gastroresistant solution/suspension of 169.4 g of polymethacrylate L100-55, 86.1 g of talc, 29 g of titanium dioxide and 15.5 g of triethyl citrate, to obtain a mini-tablet with a mean weight of 47.3 mg.

When subjected to disintegration and dissolution tests at pH 1, the tablets remain intact for at least 2 hours, with release below 1%; when subjected to the dissolution test at pH≥6.4 they exhibit the following release profile: not more than 50% after 1 hour, at pH 7.2 not more than 60% after 1 hour, and not more than 70% after 2 hours; not more than 80% after 6 hours; not more than 85% after 8 hours; and 100% after 10 hours.

### EXAMPLE 29

250 g of losartan is loaded into a granulator with 475 g of lactose and 1.8 Kg of microcrystalline cellulose. 125 g of hydroxypropyl methylcellulose (HPMC K4M), 125 g of hydroxypropyl methylcellulose (HPMC K100 M), 2.25 g of polymethacrylate L 100 and 2.25 g of polymethacrylate S 100 are added in sequence to the resulting mixture. The ingredients are mixed until a homogeneous dispersion of active ingredient in the matrices is obtained. 22.5 g of talc and 13 g of magnesium stearate are then added in sequence. The mixture is then homogenised for at least 15 minutes. This mixture will form part of the first layer of the controlled-release mini-tablet.

250 g of losartan is loaded into a second granulator. 60 g of microcrystalline cellulose, 475 g of lactose monohydrate, 225 g of crospovidone, 225 g of croscarmellose, 13 g of magnesium stearate and 27 g of talc are added and homogeneously mixed. The mixture is then homogenised for at least 15 minutes. This mixture will form part of the second, immediate-release layer of the tablet. The two separate mixtures are then compressed to obtain a double-layer mini-tablet weighing 45.15 mg. The resulting tablets are film-coated with a gastroresistant solution/suspension of 84.7 g of polymethacrylate L100, 84.7 g of polymethacrylate S 100, 83.85 g of talc, 29 g of titanium dioxide and 15.5 g of triethyl citrate, to obtain a mini-tablet with a mean weight of 47.3 mg.

When subjected to disintegration and dissolution tests at pH 1, the tablets remain intact for at least 2 hours, with release below 1%; when subjected to the dissolution test at pH≥6.4 they exhibit the following release profile: not more than 15% after 1 hour, at pH 7.2 not more than 60% after 1 hour, and not more than 70% after 2 hours; not more than 80% after 6 hours; not more than 85% after 8 hours; and 100% after 10 hours.

### EXAMPLE 30

250 g of losartan is loaded into a granulator with 475 g of lactose and 1.8 Kg of microcrystalline cellulose. 125 g of hydroxypropyl methylcellulose (HPMC K4 M), 125 g of hydroxypropyl methylcellulose (HPMC K100 M) and 45 g of polymethacrylate L 100-55 are added in sequence to the resulting mixture. The ingredients are mixed until a homogeneous dispersion of active ingredient in the matrices is obtained, 22.5 g of talc and 13 g of magnesium stearate are then added in sequence, and the mixture is homogenised for at least 15 minutes. This mixture will form part of the first layer of the controlled-release mini-tablet.

250 g of losartan is loaded into a second granulator. 60 g of microcrystalline cellulose, 475 g of lactose monohydrate, 225 g of crospovidone, 225 g of croscarmellose, 13 g of magnesium stearate and 27 g of talc are added and homogeneously mixed. The mixture is then homogenised for at least 15 minutes. This mixture will form part of the second, immediate-release layer of the tablet. The two separate mixtures are then compressed to obtain a double-layer mini-tablet weighing 44.3 mg. The resulting tablets are film-coated with an aqueous solution/suspension of 270 g of ethylcellulose, 7.75 g of triethyl citrate and 43.05 of talc, and then with a gastroresistant solution/suspension of 169.4 g of polymethacrylate L100-55, 43.05 g of talc, 29 g of titanium dioxide and 7.75 g of triethyl citrate until a mini-tablet with a mean weight of 50 mg is obtained.

When subjected to disintegration and dissolution tests at pH 1, the tablets remain intact for at least 2 hours, with release below 1%; when subjected to the dissolution test at pH≥6.4 they exhibit the following release profile: not more than 10% after 1 hour, at pH 7.2 not more than 50% after 1 hour, and not more than 70% after 2 hours; not more than 80% after 6 hours; not more than 85% after 8 hours; not more than 95% after 10 hours; and 100% after 18 hours.

### EXAMPLE 31

1.6 Kg of valsartan is loaded into a granulator with 2 Kg of lactose monohydrate, 1.0 Kg of microcrystalline cellulose, 450 g of hydroxypropyl methylcellulose (HPMC K4M), 450 g of hydroxypropyl methylcellulose (HPMC K 100M) and 9 g of polymethacrylate L100-55. The ingredients are mixed until a homogeneous dispersion of active ingredient in the matrices is obtained; 26 g of magnesium stearate and 45 g of talc are then added in sequence. The mixture is then homogenised for at least 15 minutes. This mixture is then compressed to obtain a tablet weighing 463 mg. The resulting tablets are then film-coated with a gastroresistant solution/suspension based on 169.4 g polymethacrylate L100-55, 86.1 g of talc, 29 g of titanium dioxide and 15.5 g of triethyl citrate, to obtain a tablet with a mean weight of 593 mg.

When subjected to disintegration and dissolution tests at pH 1, the tablets remain intact for at least 2 hours, with release below 1%; when subjected to the dissolution test at pH≥6.4 they exhibit the following release profile: not more than 20% after 1 hour, at pH 7.2 not more than 30% after 1 hour, not more than 60% after 2 hours; the value must be >80% after 6 hours; and 100% after 10 hours.

### EXAMPLE 32

320 g of candesartan is loaded into a granulator with 1.32 Kg of lactose monohydrate, 450 g of microcrystalline cellulose, 450 g of hydroxypropyl methylcellulose (HPMC K4M), 450 g of hydroxypropyl methylcellulose (HPMC K100M), 4.5 g of polymethacrylate RL100 and 4.5 g of polymethacrylate RS100. The ingredients are mixed until a homogeneous dispersion of active ingredient in the matrices is obtained; 26 g of magnesium stearate and 45 g of talc are then added in sequence. The mixture is then homogenised for at least 15 minutes. This mixture is then compressed to obtain a tablet weighing 307 mg. The resulting tablets are then film-coated with a solution/suspension based on 169.4 g of hydroxypropyl methylcellulose E5 Premium, 86 g of talc, 29 g of titanium dioxide and 15.5 g of triethyl citrate, to obtain a tablet with a mean weight of 337 mg.

When subjected to a dissolution test in water, the tablets exhibit the following release profile: the value must be >80% after 6 hours and 100% after 10 hours.

### EXAMPLE 33

400 g of olmesartan is loaded into a mixer/granulator with 1.4 Kg of lactose monohydrate, 450 g of microcrystalline cellulose, 500 g of hydroxypropyl methylcellulose (HPMC K4M), 400 g of hydroxypropyl methylcellulose (HPMC K100M) and 9 g of polymethacrylate L100-55. The ingredients are mixed until a homogeneous dispersion of active ingredient in the matrices is obtained; 26 g of magnesium stearate and 45 g of talc are then added in sequence. The mixture is then homogenised for at least 15 minutes. This mixture is then compressed to obtain a tablet weighing 323 mg. The resulting tablets are film-coated with a gastroresistant solution/suspension based on 169.4 g of polymethacrylate L100-55, 43 g of talc, 14.5 g of titanium dioxide and 7.75 g of triethyl citrate, and then with a solution/suspension of 100 g of ethylcellulose, 43 g of talc, 14.5 g of titanium dioxide and 7.75 g of triethyl citrate, to obtain a tablet with a mean weight of 363 mg.

When subjected to disintegration and dissolution tests at pH 1, the tablets remain intact for at least 2 hours, with release below 1%; when subjected to the dissolution test at pH≥6.4 they exhibit the following release profile: not more than 5% after 1 hour, at pH 7.2 not more than 20% after 1 hour, and not more than 40% after 2 hours; the value must be >80% after 6 hours; and 100% after 18 hours.

### EXAMPLE 34

1.5 Kg of irbesartan is loaded into a granulator with 1.5 Kg of lactose and 337.5 g of microcrystalline cellulose. 225 g of hydroxypropyl methylcellulose (HPMC K4M), 225 g of hydroxypropyl methylcellulose (HPMC K100 M) and 4.5 g of polymethacrylate L 100-55 are added in sequence to the resulting mixture; and the ingredients are mixed until a homogeneous dispersion of active ingredient in the matrices is obtained. 22.5 g of talc and 13 g of magnesium stearate are then added in sequence. The mixture is then homogenised for at least 15 minutes. This mixture will form part of the first, controlled-release layer of the tablet.

1.5 Kg of irbesartan is loaded into a second granulator. 112.5 g of microcrystalline cellulose, 500 g of lactose monohydrate, 225 g of crospovidone, 225 g of croscarmellose, 13 g of magnesium stearate and 27 g of talc are added and homogeneously mixed. The mixture is then homogenised for at least 15 minutes. This mixture will form part of the second, immediate-release layer of the tablet. The two separate mixtures are then compressed to obtain a double-layer tablet weighing 643 mg.

When subjected to disintegration and dissolution tests at pH 1, the tablets remain intact for at least 2 hours, with release below 1%; when subjected to the dissolution test at pH≥6.4 they exhibit the following release profile: not more than 15% after 1 hour, at pH 7.2 not more than 50% after 1 hour, and not more than 60% after 2 hours; not more than 75% after 6 hours; less than 85% after 8 hours; less than 95% after 10 hours; and 100% after 18 hours.

### EXAMPLE 35

400 g of telmisartan is loaded into a granulator with 1.5 Kg of lactose and 337.5 g of microcrystalline cellulose. 225 g of hydroxypropyl methylcellulose (HPMC K100 1v), 225 g of hydroxypropyl methylcellulose (HPMC K15 M), 2.25 g of polymethacrylate RL 100 and 2.25 g of polymethacrylate RS 100 are added in sequence to the resulting mixture, and the ingredients are mixed until a homogeneous dispersion of active ingredient in the matrices is obtained. 22.5 g of talc and 13 g of magnesium stearate are then added in sequence. The mixture is then homogenised for at least 15 minutes. This mixture will form part of the first, controlled-release layer of the tablet.

400 g of telmisartan is loaded into a second granulator. 112.5 g of microcrystalline cellulose, 500 g of lactose monohydrate, 225 g of crospovidone, 225 g of croscarmellose, 13 g of magnesium stearate and 27 g of talc are added and homogeneously mixed. The mixture is then homogenised for at least 15 minutes. This mixture will form part of the second, immediate-release layer of the tablet. The two separate mixtures are then compressed to obtain a double-layer tablet weighing 423 mg. The resulting tablets are then film-coated with a solution/suspension based on 169.4 g of hydroxypropyl methylcellulose E5 P, 86 g of talc, 29 g of titanium dioxide and 15.5 g of triethyl citrate, to obtain a tablet with a mean weight of 453 mg.

When subjected to dissolution tests in water, the tablets exhibit the following release profile: not more than 25% after 1 hour, not more than 55% after 2 hours; the value must be >85% after 6 hours; and 100% after 10 hours.

### EXAMPLE 36

300 g of eprosartan is loaded into a granulator with 1.5 Kg of lactose and 337.5 g of microcrystalline cellulose. 225 g of hydroxypropyl methylcellulose (HPMC K4M), 225 g of hydroxypropyl methylcellulose (HPMC K100 M) and 4.5 g of polymethacrylate

L 100-55 are added in sequence to the resulting mixture, and the ingredients are mixed until a homogeneous dispersion of active ingredient in the matrices is obtained. 22.5 g of talc and 13 g of magnesium stearate are then added in sequence. The mixture is then homogenised for at least 15 minutes. This mixture will form part of the first, controlled-release layer of the tablet.

300 g of eprosartan is loaded into a second granulator. 112.5 g of microcrystalline cellulose, 1 Kg of lactose monohydrate, 225 g of crospovidone, 225 g of croscarmellose, 13 g of magnesium stearate and 27 g of talc are added and homogeneously mixed. The mixture is then homogenised for at least 15 minutes. This mixture will form part of the second, immediate-release layer of the tablet. The two separate mixtures are then compressed to obtain a double-layer tablet weighing 993 mg. The tablets thus obtained are film-coated with a solution/suspension of 270 g of ethylcellulose, 43.05 g of talc, 14.5 g of titanium dioxide and 7.75 g of triethyl citrate, and then with a gastroresistant suspension/solution of 169.4 g of polymethacrylate L 100-55, 43.05 g of talc, 14.5 g of titanium dioxide and 7.75 g of triethyl citrate, to obtain a tablet with a mean weight of 1050 mg.

When subjected to disintegration and dissolution tests at pH 1, the tablets remain intact for at least 2 hours, with release below 1%; when subjected to the dissolution test at pH≥6.4 they exhibit the following release profile: not more than 10% after 1 hour, at pH 7.2 not more than 40% after 1 hour, and not more than 50% after 2 hours; not more than 70% after 6 hours; less than 80% after 8 hours; not more than 95% after 10 hours; and 100% after 18 hours.

The following tables summarise the qualitative and quantitative compositions of Examples 1-36.

TABLE 1
Losartan 100 mg tablet

| | F1 | F2 | F3 | F4 | F5 | F6 |
|---|---|---|---|---|---|---|
| Ingredients | | | | | | |
| of MR core | | | | | | |
| Losartan | 100 | 100 | 100 | 50 | 50 | 50 |
| potassium | | | | | | |
| Lactose | 200 | 200 | 200 | 150 | 150 | 150 |
| monohydrate | | | | | | |
| Microcrystalline | 45 | 45 | 45 | 33.75 | 33.75 | 33.75 |
| cellulose | | | | | | |
| HPMC 100 lv | | | | | 22.5 | |
| HPMC K4 M | 45 | 45 | 45 | 22.5 | | 22.5 |
| HPMC K15 M | | | | | 22.5 | |
| HPMC K100 M | 45 | 45 | 45 | 22.5 | | 22.5 |
| Eudragit L100/55 | 0.9 | | 0.9 | 0.45 | | 0.45 |
| Eudragit L 100 | | 0.45 | | | 0.225 | |
| Eudragit S 100 | | 0.45 | | | 0.225 | |
| Talc | 4.5 | 4.5 | 4.5 | 2.25 | 2.25 | 2.25 |
| Mg stearate | 2.6 | 2.6 | 2.6 | 1.3 | 1.3 | 1.3 |
| | 443 | 443 | 443 | 282.25 | 282.25 | 282.25 |
| Ingredients | | | | | | |
| of IR core | | | | | | |
| Losartan | | | | 50 | 50 | 50 |
| potassium | | | | | | |
| Lactose | | | | 50 | 50 | 50 |
| monohydrate | | | | | | |
| Microcrystalline | | | | 11.25 | 11.25 | 11.25 |
| cellulose | | | | | | |
| Crosslinked PVP | | | | 22.5 | 22.5 | 22.5 |
| AcDisol | | | | 22.5 | 22.5 | 22.5 |
| Talc | | | | 2.7 | 2.7 | 2.7 |
| Mg stearate | | | | 1.3 | 1.3 | 1.3 |
| Total | | | | 160.25 | 160.25 | 160.25 |
| Film-coating | | | | | | |
| ingredients | | | | | | |
| Talc | 8.61 | 8.61 | 8.61 | 8.61 | 8.61 | 8.61 |
| Eudragit L100/55 | 16.94 | | 16.94 | 16.94 | | 16.94 |
| Eudragit L 100 | | 8.47 | | | 8.47 | |
| Eudragit S 100 | | 8.47 | | | 8.47 | |
| Ethylcellulose | | | 27 | | | 27 |
| Titanium dioxide | 2.90 | 2.90 | 2.90 | 2.90 | 2.90 | 2.90 |
| Triethyl citrate | 1.55 | 1.55 | 1.55 | 1.55 | 1.55 | 1.55 |
| Total | 30 | 30 | 57 | 30 | 30 | 57 |
| Grand total | 473 | 473 | 500 | 473 | 473 | 500 |

TABLE 2
Losartan 50 mg tablet

| | F7 | F8 | F9 | F10 | F11 | F12 |
|---|---|---|---|---|---|---|
| Ingredients | | | | | | |
| of MR core | | | | | | |
| Losartan | 50 | 50 | 50 | 25 | 25 | 25 |
| potassium | | | | | | |
| Lactose | 100 | 100 | 100 | 75 | 75 | 75 |
| monohydrate | | | | | | |
| Microcrystalline | 22.5 | 22.5 | 22.5 | 17 | 17 | 17 |
| cellulose | | | | | | |
| HPMC K4 M | 12.5 | 12.5 | 12.5 | 11 | 11 | 11 |
| HPMC K100 M | 32.5 | 32.5 | 32.5 | 16 | 16 | 16 |
| Eudragit L100/55 | 0.45 | | 0.45 | 0.2 | | 0.2 |
| Eudragit L 100 | | 0.225 | | | 0.1 | |
| Eudragit S 100 | | 0.225 | | | 0.1 | |
| Talc | 2.25 | 2.25 | 2.25 | 1.1 | 1.1 | 1.1 |
| Mg stearate | 1.3 | 1.3 | 1.3 | 0.7 | 0.7 | 0.7 |
| | 221.5 | 221.5 | 221.5 | 146 | 146 | 146 |
| Ingredients | | | | | | |
| of IR core | | | | | | |
| Losartan | | | | 25 | 25 | 25 |
| potassium | | | | | | |
| Lactose | | | | 25 | 25 | 25 |
| monohydrate | | | | | | |
| Microcrystalline | | | | 5 | 5 | 5 |
| cellulose | | | | | | |
| Crosslinked PVP | | | | 11 | 11 | 11 |
| AcDisol | | | | 11 | 11 | 11 |
| Talc | | | | 2.2 | 2.2 | 2.2 |
| Mg stearate | | | | 0.8 | 0.8 | 0.8 |
| Total | | | | 80 | 80 | 80 |
| Film-coating | | | | | | |
| ingredients | | | | | | |
| Talc | 5.74 | 5.74 | 5.74 | 5.74 | 5.74 | 5.74 |
| Eudragit L100/55 | 12.29 | | 12.29 | 12.29 | | 12.29 |
| Eudragit L 100 | | 6.145 | | | 6.145 | |
| Eudragit S 100 | | 6.145 | | | 6.145 | |
| Ethylcellulose | | | 12 | | | 12 |
| Titanium dioxide | 1.93 | 1.93 | 1.93 | 1.93 | 1.93 | 1.93 |
| Triethyl citrate | 1.54 | 1.54 | 1.54 | 1.54 | 1.54 | 1.54 |
| Total | 21.5 | 21.5 | 33.5 | 21.5 | 21.5 | 33.5 |
| Grand total | 243 | 243 | 255 | 247.5 | 247.5 | 259.5 |

TABLE 3
Losartan 25 mg tablet

| | F13 | F14 | F15 | F16 | F17 | F18 |
|---|---|---|---|---|---|---|
| Ingredients | | | | | | |
| of MR core | | | | | | |
| Losartan | 25 | 25 | 25 | 12.5 | 12.5 | 12.5 |
| potassium | | | | | | |
| Lactose | 150 | 150 | 150 | 93.75 | 93.75 | 93.75 |
| monohydrate | | | | | | |
| Microcrystalline | 22.5 | 22.5 | 22.5 | 16 | 16 | 16 |
| cellulose | | | | | | |
| HPMC K4 M | 12.5 | 12.5 | 12.5 | 11 | 11 | 11 |
| HPMC K100 M | 32.5 | 32.5 | 32.5 | 16 | 16 | 16 |
| Eudragit L100/55 | 0.45 | | 0.45 | 0.2 | | 0.2 |
| Eudragit L 100 | | 0.225 | | | 0.1 | |
| Eudragit S 100 | | 0.225 | | | 0.1 | |
| Talc | 2.25 | 2.25 | 2.25 | 1.1 | 1.1 | 1.1 |
| Mg stearate | 1.3 | 1.3 | 1.3 | 0.7 | 0.7 | 0.7 |
| Ingredients | 221.5 | 221.5 | 221.5 | 151.25 | 151.25 | 151.25 |
| of IR core | | | | | | |
| Losartan | | | | 12.5 | 12.5 | 12.5 |
| potassium | | | | | | |
| Lactose | | | | 31.25 | 31.25 | 31.25 |
| monohydrate | | | | | | |
| Microcrystalline | | | | 6 | 6 | 6 |
| cellulose | | | | | | |
| Crosslinked PVP | | | | 11 | 11 | 11 |
| AcDisol | | | | 11 | 11 | 11 |
| Talc | | | | 2.2 | 2.2 | 2.2 |
| Mg stearate | | | | 0.8 | 0.8 | 0.8 |
| Total | | | | 74.75 | 74.75 | 74.75 |
| Film-coating | | | | | | |
| ingredients | | | | | | |
| Talc | 5.74 | 5.74 | 5.74 | 5.74 | 5.74 | 5.74 |
| Eudragit L100/55 | 12.29 | | 12.29 | 12.29 | | 12.29 |
| Eudragit L 100 | | 6.145 | | | 6.145 | |
| Eudragit S 100 | | 6.145 | | | 6.145 | |
| Ethylcellulose | | | 12 | | | 12 |
| Titanium dioxide | 1.93 | 1.93 | 1.93 | 1.93 | 1.93 | 1.93 |
| Triethyl citrate | 1.54 | 1.54 | 1.54 | 1.54 | 1.54 | 1.54 |
| Total | 21.5 | 21.5 | 33.5 | 21.5 | 21.5 | 33.5 |
| Grand total | 243 | 243 | 255 | 247.5 | 247.5 | 259.5 |

TABLE 4
Losartan 47.3 mg mini-tablets (10 mg losartan per mini-tablet = 10
mini-tablets to give 100 mg of active ingredient = 5 mini-tablets to give
50 mg of active ingredient).

| | F19 | F20 | F21 | F22 | F23 | F24 |
|---|---|---|---|---|---|---|
| Ingredients of MR MiniCore | | | | | | |
| Losartan potassium | 10 | 10 | 10 | 5 | 5 | 5 |
| Microcrystalline cellulose | 20 | 20 | 20 | 15 | 15 | 15 |
| Lactose monohydrate | 4.5 | 4.5 | 4.5 | 2.25 | 2.25 | 2.25 |
| HPMC k 100 lv | | | | | | 2.25 |
| HPMC K4 M | 4.5 | 4.5 | 4.5 | 2.25 | 2.25 | |
| HPMC K15 M | | | | | | 2.25 |
| HPMC K100 M | 4.5 | 4.5 | 4.5 | 2.25 | 2.25 | |
| Eudragit L100/55 | 0.09 | | 0.09 | 0.045 | | 0.045 |
| Eudragit L 100 | | 0.045 | | | | |
| Eudragit S 100 | | 0.045 | | | | |
| Eudragit RL 100 | | | | | 0.0225 | |
| Eudragit RS 100 | | | | | 0.0225 | |
| Shellac | | | | | 0.0225 | |
| Talc | 0.45 | 0.45 | 0.45 | 0.225 | 0.225 | 0.225 |
| Mg stearate | 0.26 | 0.26 | 0.26 | 0.13 | 0.13 | 0.13 |
| | 44.3 | 44.3 | 44.3 | 27.15 | 27.15 | 27.15 |
| Ingredients of IR core | | | | | | |
| Losartan potassium | | | | 5 | 5 | 5 |
| Microcrystalline cellulose | | | | 5 | 5 | 5 |
| Lactose monohydrate | | | | 2.25 | 2.25 | 2.25 |
| Crosslinked PVP | | | | 2.25 | 2.25 | 2.25 |
| AcDisol | | | | 2.25 | 2.25 | 2.25 |
| Talc | | | | 0.27 | 0.27 | 0.27 |
| Mg stearate | | | | 0.13 | 0.13 | 0.13 |
| Total | | | | 17.5 | 17.5 | 17.5 |
| Film-coating ingredients | | | | | | |
| Talc | 0.861 | 0.861 | 0.861 | 0.861 | 0.861 | 0.861 |
| Eudragit L100/55 | 1.694 | | 1.694 | 1.694 | | 1.694 |
| Eudragit L 100 | | 0.847 | | | | |
| Eudragit S 100 | | 0.847 | | | | |
| Shellac | | | | | 1.694 | |
| Ethylcellulose | | | 2.7 | | | 27 |
| Titanium dioxide | 0.290 | 0.290 | 0.290 | 0.290 | 0.290 | 0.290 |
| Triethyl citrate | 0.155 | 0.155 | 0.155 | 0.155 | 0.155 | 0.155 |
| Total | 3.0 | 3.0 | 5.7 | 3.0 | 3.0 | 5.7 |
| Grand total | 47.3 | 47.3 | 50.0 | 47.3 | 47.3 | 50.0 |

TABLE 5
Losartan 47.3 mg mini-tablets (5 mg losartan per mini-tablet = 5
mini-tablets to give 25 mg of active ingredient).

| | F25 | F26 | F27 | F28 | F29 | F30 |
|---|---|---|---|---|---|---|
| Ingredients of MR MiniCore | | | | | | |
| Losartan potassium | 5 | 5 | 5 | 2.5 | 2.5 | 2.5 |
| Microcrystalline cellulose | 24 | 24 | 24 | 18 | 18 | 18 |
| Lactose monohydrate | 9.5 | 9.5 | 9.5 | 4.75 | 4.75 | 4.75 |
| HPMC 100 lv | | | | | | |
| HPMC K4 M | 2.5 | 2.5 | 2.5 | 1.25 | 1.25 | 1.25 |
| HPMC K15 M | | | | | | |
| HPMC K100 M | 2.5 | 2.5 | 2.5 | 1.25 | 1.25 | 1.25 |
| Eudragit L100/55 | 0.09 | | 0.09 | 0.045 | | 0.045 |
| Eudragit L 100 | | 0.045 | | | 0.0225 | |
| Eudragit S 100 | | 0.045 | | | 0.0225 | |
| Talc | 0.45 | 0.45 | 0.45 | 0.225 | 0.225 | 0.225 |
| Mg stearate | 0.26 | 0.26 | 0.26 | 0.13 | 0.13 | 0.13 |
| | 44.3 | 44.3 | 44.3 | 28.15 | 28.15 | 28.15 |
| Ingredients of IR core | | | | | | |
| Losartan potassium | | | | 2.5 | 2.5 | 2.5 |
| Microcrystalline cellulose | | | | 6 | 6 | 6 |
| Lactose monohydrate | | | | 4.75 | 4.75 | 4.75 |
| Crosslinked PVP | | | | 2.25 | 2.25 | 2.25 |
| AcDisol | | | | 2.25 | 2.25 | 2.25 |
| Talc | | | | 0.27 | 0.27 | 0.27 |
| Mg stearate | | | | 0.13 | 0.13 | 0.13 |
| Total | | | | 16.15 | 16.15 | 16.15 |
| Film-coating ingredients | | | | | | |
| Talc | 0.861 | 0.861 | 0.861 | 0.861 | 0.861 | 0.861 |
| Eudragit L100/55 | 1.694 | | 1.694 | 1.694 | | 1.694 |
| Eudragit L 100 | | 0.847 | | | 0.847 | |
| Eudragit S 100 | | 0.847 | | | 0.847 | |
| Ethylcellulose | | | 2.7 | | | 27 |
| Titanium dioxide | 0.290 | 0.290 | 0.290 | 0.290 | 0.290 | 0.290 |
| Triethyl citrate | 0.155 | 0.155 | 0.155 | 0.155 | 0.155 | 0.155 |
| Total | 3.0 | 3.0 | 5.7 | 3.0 | 3.0 | 5.7 |
| Grand total | 47.3 | 47.3 | 50.0 | 47.3 | 47.3 | 50.0 |

TABLE 6
SARTAN tablets

| | F31 | F32 | F33 | F34 | F35 | F36 |
|---|---|---|---|---|---|---|
| Ingredients | | | | | | |
| of MR core | | | | | | |
| Valsartan | 160 | | | | | |
| Candesartan | | 32 | | | | |
| Olmesartan | | | 40 | | | |
| Irbesartan | | | | 150 | | |
| Telmisartan | | | | | 40 | |
| Eprosartan | | | | | | 300 |
| Lactose hydrate. | 200 | 132 | 140 | 150 | 150 | 150 |
| Microcrystalline | 105 | 45 | 45 | 33.75 | 33.75 | 33.75 |
| cellulose | | | | | | |
| HPMC 100 lv | | | | | 22.5 | |
| HPMC K4 M | 45 | 45 | 50 | 22.5 | | 22.5 |
| HPMC K15 M | | | | | 22.5 | |
| HPMC K100 M | 45 | 45 | 40 | 22.5 | | 22.5 |
| Eudragit L100/55 | 0.9 | | 0.9 | 0.45 | | 0.45 |
| Eudragit RL 100 | | 0.45 | | | 0.225 | |
| Eudragit RS 100 | | 0.45 | | | 0.225 | |
| Talc | 4.5 | 4.5 | 4.5 | 2.25 | 2.25 | 2.25 |
| Mg stearate | 2.6 | 2.6 | 2.6 | 1.3 | 1.3 | 1.3 |
| | 563 | 307 | 323 | 382.75 | 272.75 | 532.75 |
| Ingredients | | | | | | |
| of IR core | | | | | | |
| Irbesartan | | | | 150 | | |
| Telmisartan | | | | | 40 | |
| Eprosartan | | | | | | 300 |
| Lactose Hydrate | | | | 50 | 50 | 100 |
| Microcrystalline | | | | 11.25 | 11.25 | 11.25 |
| cellulose | | | | | | |
| Crosslinked PVP | | | | 22.5 | 22.5 | 22.5 |
| AcDisol | | | | 22.5 | 22.5 | 22.5 |
| Talc | | | | 2.7 | 2.7 | 2.7 |
| Mg stearate | | | | 1.3 | 1.3 | 1.3 |
| Total | | | | 260.25 | 150.25 | 460.25 |
| Coating | | | | | | |
| ingredients | | | | | | |
| Talc | 8.61 | 8.61 | 8.61 | 8.61 | 8.61 | 8.61 |
| Eudragit L100/ 55 | 16.94 | | 16.94 | 16.94 | | 16.94 |
| HPMC E5P | | 16.94 | | | 16.94 | |
| Ethylcellulose | | | 10 | | | 27 |
| Titanium dioxide | 2.90 | 2.90 | 2.90 | 2.90 | 2.90 | 2.90 |
| Triethyl citrate | 1.55 | 1.55 | 1.55 | 1.55 | 1.55 | 1.55 |
| Total | 30 | 30 | 40 | 30 | 30 | 57 |
| Grand total | 593 | 337 | 363 | 673 | 453 | 1050 |

1. A controlled-release solid oral pharmaceutical composition comprising a core containing a sartan and an outer coating of said core, wherein:
a) the core comprises:
(i) a monolithic matrix containing a sartan, at least one hydroxypropyl methylcellulose having a viscosity ranging between 3 and 5000 mPa·s 2% in H₂O at 20° C., at least one hydroxypropyl methylcellulose having a viscosity ranging between 13500 and 280000 mPa·s 2% in H₂O at 20° C., at least one or more methacrylic polymers/copolymers and/or shellac, cellulose acetate phthalate and/or hydroxypropyl methylcellulose acetate succinate, or
(ii) a monolithic matrix as defined above adjacent to an immediate-release layer containing a sartan;
b) the outer coating comprises a layer comprising ethylcellulose or a gastro-resistant layer or a layer comprising ethylcellulose which in turn is coated with gastroresistant polymers.

2. A composition as claimed in claim 1 wherein the core comprises a monolithic matrix as defined in claim 1, point (i).

3. A composition as claimed in claim 1 wherein the core comprises a monolithic matrix as defined in claim 1, adjacent to an immediate-release layer of sartan.

4. A composition as claimed in claim 1 wherein the outer coating comprises a layer comprising ethylcellulose.

5. A composition as claimed in claim 1 wherein the outer coating comprises a layer comprising ethylcellulose coated with gastroresistant polymers.

6. A composition as claimed in claim 1 wherein the outer coating comprises a gastroresistant layer.

7. A composition as claimed in claim 1 wherein the acrylic/methacrylic polymers or copolymers are selected from pH-independent methacrylic ester copolymers, pH-independent ammonium alkyl methacrylate copolymers; amino alkyl methacrylate copolymers soluble up to pH 5.0, methacrylic acid copolymers soluble at pH≥5.5, methacrylic acid copolymers soluble at pH 6.0-7.0; and pH-dependent methacrylic acid copolymers soluble at pH≥7.0.

8. A composition as claimed in claim 1 wherein the monolithic matrix comprises shellac.

9. A composition as claimed in claim 1 wherein the gastroresistant coating comprises pH-dependent methacrylic acid copolymers soluble at pH≥5.5; pH-dependent methacrylic acid copolymers soluble at pH 6.0-7.0; pH-dependent methacrylic acid copolymers soluble at pH≥7.0; shellac; cellulose acetate phthalate; and/or hydroxypropyl methylcellulose acetate succinate.

10. A composition as claimed in claim 1 wherein the hydroxypropyl methylcellulose having a viscosity ranging between 3 and 5000 mPa·s 2% in H₂O at 20° C. constitutes 1 to 20% of the weight of the core, the hydroxypropyl methylcellulose having a viscosity ranging between 13500 and 280000 mPa·s 2% in H₂O at 20° C. constitutes 1 to 20% of the weight of the matrix, and the methacrylic polymer/copolymer constitutes 0.1 to 2% of the weight of the core.

11. A composition as claimed in claim 1 wherein ethylcellulose is present in percentages ranging from 1 to 20% of the weight of the core.

12. A composition as claimed in claim 1 wherein the sartan is losartan, valsartan, irbesartan or olmesartan.

13. A composition as claimed in claim 1, wherein the sartan is losartan.
